(19) United States
(12) Patent Application Publication
(10) Pub. No.: US 2003/0147896 A1
(43) Pub. Date: Aug. 7, 2003

(54) Herbal fomulation

(76) Inventor: Ranjitsinh Solanki, Sania-Kande (IN)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 10/257,082

(22) PCT Filed: Jul. 10, 2002
(86) PCT No.: PCT/IB02/02699

(30) Foreign Application Priority Data
Jul. 11, 2001 (GB) 0116942.4

(51) Int. Cl. A61K 39/395

(52) U.S. Cl. 424/181.1

(57) ABSTRACT

A pharmaceutical or medicinal preparation which comprises a mixture of the following seven herbs: Tinospora cordifolia, Chlorphyton borivilianum, Curcuma longa, Asparagus racemosus, Hygrophila auriculata, Achyranthus aspera and Elephantopus scaber, or a mixture of the active ingredients that have been extracted from those herbs or chemically synthesised. The herbal formulation of the invention is effective for the treatment of cancer, in particular myelomas.

### TECHNICAL FIELD

This invention relates to a new herbal formulation which has been found to be effective for the treatment of cancer. More particularly, the formulation can be used to treat myelomas.

The conventional treatment of cancer comprises surgery, chemotherapy and/or radiotherapy. The drugs given during chemotherapy are of necessity very powerful and, in consequence, can have serious and undesirable side-effects. There is therefore a need for improved pharmaceutical or medicinal preparations for use in the treatment of cancer. It is the objet of this invention to provide such a product.

According to this invention there is provided a pharmaceutical or medicinal preparation which comprises a mixture of the following seven herbs: Tinospora cordifolia, Chlorphyton borivilianum, Curcuma longa, Asparagus racemosus, Hygrophila auriculata, Achyranthus aspera and Elephantopus scaber, or a mixture of the active ingredients that have been extracted from those herbs or chemically synthesized. This product has been found by the inventor to be particularly effective for the treatment of all myelomas, including both solitary and multiple myelomas. The preparation is preferably formulated for administration to patients as a liquid or syrup, but could also be administered as a capsule or tablet.

The ingredients for a typical herbal formulation according to this invention are set out in Table I. It should be appreciated that the proportions of the individual herbs may be varied and the figures quoted in Table I are by way of illustration only. In particular, the proportions of one or more of the components may be varied in order to optimize the pharmacological effects produced by the formulation to suit the specific needs of patients being treated.

It is an important feature of the product of the present invention that it contains a mixture of herbs, or extracts from herbs, rather than being based on a single herb. A synergistic effect has been noticed between the various ingredients. This synergistic activity is surprising and unexpected. The activities of similar herbs are combined to optimize and enhance the pharmacological effects without increasing the adverse toxic reactions (which becomes a distinct possibility if the herbs are used singly in a concentration of 100%). The advantage of a multi-drug regimen also lies in the fact that the possibility of development of drug resistance is minimized.

Preliminary clinical trials of the product of this invention have produced definite clinical evidence of improvement in the condition of patients suffering from myelomas. These improvements include:

i) reduction in the number of myeloma cells from the bone marrow, and other bony as well as soft tissue tumour infiltrates,

ii) improvement in radiological parameters, such as osteolysis,

iii) improvement in the relevant biochemical parameters, such as a reduction in plasma globulins, and

iv) disappearance of the M-band from protein electrophoresis; and more subjectively:

i) sense of well being,

ii) improvement in appetite; and

iii) increased vigour and enthusiasm in daily activities.

The formulation of this invention is itself effective for the treatment of cancer. It may also be used as an adjuvant to conventional modes of anticancer therapy, namely radiotherapy and/or chemotherapy. The formulation may be presented as a dietary supplement for patients diagnosed as having any type of cancer. It may also be used to create a sense of general well being and to increase the vitality in patients diagnosed as having any type of cancer, to increase the appetite, restore health and increase the lifespan of patients diagnosed as having any type of cancer, to improve the ambulatory capacity in patients diagnosed as having any type of cancer, to activate the nervous system, prevent degenerative changes, stimulate regeneration and improve the psychological status in patients diagnosed as having any type of cancer; and to stimulate metabolism, accelerate anabolism, promote catabolism thereby flushing the body of toxic metabolites and reducing the side effects of chemotherapy and radiotherapy. The hepatic clearance of substances like iron and ferritin in cases of thalassemia is also improved, thereby reducing the iron overload in such cases.

The manufacture of a product according to the present invention will now be illustrated by the following example. However, it will be appreciated that the active ingredients may be chemically synthesized as an alternative to being extract from the natural herbs.

### EXAMPLE

Method of Extraction

Each of the herbal components of the formulation were de-seeded (wherever required), ground finely to powder form and then submitted individually to conventional solvent extraction methods.

By way of illustration only, the extraction can be performed by using volatile freon gas. This process has the advantage of being fast and also has the ability to preserve the active chemicals (alkaloids, non-alkaloids, electrolytes, minerals, etc.) in their natural form (as it does not involve heating and denaturation at any stage of the process). Freon, being a highly volatile compound with its boiling point at −21° C., evaporates totally after extraction, yielding an ultrapure concentrate of the chemicals. The chemicals are thereafter diluted appropriately and mixed in the proportions mentioned in Table 1.

Preliminary Clinical Data

Case 1

The patient was a 53 year old male complaining of backache, nausea, fatigue, muscle weakness and fever. His clinical history suggested some haematological malignancies. Clinical investigation and confirmatory pathological tests (serum protein electrophoresis, urine protein electrophoresis, cytological examination of bone marrow aspiration and urine profile) were performed. The patient was diagnosed as having light chain diseases/multiple myeloma (Bence Jones Protein).

The patient began taking the herbal formulation of the present invention on Aug. 16, 2000. The formulation as defined in Table 1 was administered to the patient in the form of a liquid at a dosage of 4 mg/kg body weight per day until February 2001. The patient tolerated the therapy very well and no adverse effects were noted. The clinical condition of the patient was found to be improved in that i) there was no evidence of paraproteinaemia, ii) the urine profile suggested an improvement and iii) the haematological profile also showed an improvement. Furthermore, and on a subjective level, the patient was found to have a sense of well being, made no complaint of backache, nausea, or fatigue and showed an increased vigour and enthusiasm in daily activities.

Case 2

A 92 year old patient who was diagnosed as a case of multiple myeloma with renal failure 15 years ago and who was undergoing regular dialysis, was put on herbal therapy in August 2001. Since then, this serum creatinine and urea levels fell steadily even during the interval periods when he was not undergoing dialysis. In fact, the interval between dialysis sessions increased over the last six months. His calcium loss through urine also reduced considerably. The patient also had an improved appetite and improved enthusiasm.

These findings are considered to show that the herbal formulations of this invention has a beneficial effect of patient suffering from myeloma. The benefits include an improved immunomodulatory effect, anti-depressant effects, improved red blood cell count, help to the kidneys in filtering excess proteins and calcium, the destruction of malignant cells and without damaging or otherwise affecting healthy cells, the prevention of excess antibody proteins and calcium and, finally, the production of no adverse side effects.

TABLE 1
Polyherbal formulation for Multiple Myelomas and other Haematological
Malignancies
Description of Ingredients

| Sr. | | Common | | | | Adverse |
|---|---|---|---|---|---|---|
| No. | Latin Binomial | Names | Distribution | Parts used | Quantity | Reactions |
| 1 | Tinospora | Tinospora, | Throughout | Stem | 35-45%, | None |
| | cordifolia | guduchi, | India in | | preferably | |
| | | Amrita | forests | | 40% | |
| 2 | Chlorphyton | Aloe | More in the | Leaf juice, | 13-17%, | None |
| | borivilianum | | drier parts of | elio | preferably 15% | |
| | | | India | | | |
| 3. | Curcuma longa | Turmeric, | Cultivated | Rhizomes | 8-12% | None |
| | | Haldi, | throughout | (dried as | preferably 10% | |
| | | Haridra | India | well as | | |
| | | | | raw) | | |
| 4. | Asparagus | Shatavari | | | 8-12% | |
| | racemosus | | | | preferably 10% | |
| 5. | Hygrophila | Kokilax | Throughout | Leaves | 8-12% | None |
| | auriculata | | India | | preferably 10% | |
| 6. | Achyranthus | Apamarg | Throughout | Whole | 8-12% | None |
| | aspera | | India along | plant | preferably 10% | |
| | | | roadsides and | | | |
| | | | waste places | | | |
| 7. | Elephantopus | Gaozaban | Few parts of | Roots, stem | 3-8% | None |
| | scaber | | India | | preferably 5% | |

1. A pharmaceutical or medicinal preparation comprising a mixture of the herbs Tinospora cordifolia, Chlorphyton borivilianum, Curcuma longa, Asparagus racemosus, Hygrophila auriculata, Achyranthus aspera and Elephantopus scaber; or a mixture of the active ingredients that have been extracted from those herbs or chemically synthesised.

2. A pharmaceutical or medicinal preparation as claimed in claim 1, for use in the treatment of cancer.

3. A pharmaceutical or medicinal preparation as claimed in claim 1, for use in the treatment of myelomas.

4. A pharmaceutical or medicinal preparation as claimed in claim 1, for use as an adjuvant to conventional modes of anticancer therapy, namely radiotherapy and/or chemotherapy.

5. A pharmaceutical or medicinal preparation as claimed in claim 1, wherein the amount of the herbs is as under:

| | |
|---|---|
| Tinospora cordifolia | 35-45% |
| Chlorphyton borivilianum | 13-17% |
| Curcuma longa | 8-12% |
| Asparagus racemosus | 8-12% |
| Hygrophila auriculata | 8-12% |
| Achyranthus aspera | 8-12% |
| Elephantopus scaber; | 3-8% |

6. A pharmaceutical or medicinal preparation as claimed in claim 5, wherein the amount of the herbs is as under:

| | |
|---|---|
| Tinospora cordifolia | 40% |
| Chlorphyton borivilianum | 15% |
| Curcuma longa | 10% |
| Asparagus racemosus | 10% |
| Hygrophila auriculata | 10% |
| Achyranthus aspera | 10% |
| Elephantopus scaber; | 5% |

7. A dietary supplement for patients diagnosed as having any type of cancer, which includes a pharmaceutical or medicinal preparation as claimed in claim 1.
